(19) United States
(12) Patent Application Publication
(10) Pub. No.: US 2006/0240098 A1
(43) Pub. Date: Oct. 26, 2006

(54) Formulations for hyperforin-enriched hypericum fractions

(75) Inventor: Trevor P. Castor, Arlington, MA (US)

Correspondence Address: BURNS & LEVINSON, LLP;(FORMERLY PERKINS SMITH & COHEN LLP), 125 SUMMER STREET, BOSTON, MA, 02110, US

(73) Assignee: Aphios Corporation, Woburn, MA (US)

(21) Appl. No.: 11/181,054

(22) Filed: Jul. 14, 2005

Related U.S. Application Data
(60) Provisional application No. 60/587,823, filed on Jul. 14, 2004.

(51) Int. Cl. A61K 36/38; A61K 9/64

(52) U.S. Cl. 424/456; 424/730

(57) ABSTRACT

St. John's Wort products which have enhanced bioactivity in a serotonin re-uptake assay and enhanced stability and bioavailability are formulated and manufactured from hyperforin-enriched Hypericum fractions made by supercritical and near critical fluids with and without polar cosolvents. These fluids are used to fractionate the biomass materials in several sequential steps. In each step, the biomass is subjected to a multiplicity of supercritical or near critical fluid extraction steps, with different solvation conditions used for each fraction. Thus, fractionation of the biomass is effected and the St. John's Wort products are manufactured. In addition to excellent overall yield, the bioactivity and stability of the St. John's Wort products manufactured from Hypericum perforatum biomass with supercritical and near critical fluids with and without polar cosolvents are significantly higher than that obtained by conventional organic phase extraction. The advanced formulation of the hyperforin-enriched Hypericum fractions includes antioxidants as oxygen scavengers to improve stability and emulsifiers such as lecithin to improve bioavailability.

### RELATED APPLICATIONS

This application claims priority to and the benefit of U.S. provisional application Ser. No. 60/587,823, filed Jul. 14, 2004.

### FIELD OF THE INVENTION

This invention relates to formulations for Hypericum fractions into St. John's Wort products. The formulations contain one or more compounds which exhibit enhanced biological activities, product stabilities and bioavailabilities. The biologically active compounds feature supercritical, critical and near critical fluids with and without polar cosolvents as well as antioxidants, emulsifiers and other excipients.

### BACKGROUND OF THE INVENTION

St. John's Wort (Hypericum perforatum L.) is a bushy perennial with yellow flowers, which blooms around St. John the Baptist's day in June. Commercial products are derived from the dried flowering tops or aerial parts of Hypericum perforatum L.; these parts are harvested shortly before or during the flowering period. Hypericum preparations include the dried herb (chopped or powdered), alcoholic extract, oil, and tincture. Hypericum contains some very common plant constituents including flavonoid derivatives such as rutin, biflavonoids such as amentoflavone, and essential oils. Active ingredients of Hypericum, which are specific to St. John's Wort (SJW), include the naphthodianthrones, such as cyclopseudohypericin, hypericin, hyperforin, isohypericin, photohypericin and psuedohypericin.

In folk and traditional systems of medicine, various species of Hypericum have been used orally to treat anxiety, bedwetting, dyspepsia, excitability, exhaustion, fibrositis, gastritis, gout, hemorrhage, pulmonary complaints, rheumatism, sciatica, and swelling. Of all the medical uses of SJW, the use of Hypericum extracts for the treatment of mild to moderate depression is the most extensive. Standardized extracts of St. John's Wort are sold in pharmacies throughout Europe, and are in fact among the most popular OTC “phytomedicines” sold in EEC countries. Among the most widely prescribed antidepressants in the United States are Prozac from Eli Lilly and Zoloft from Pfizer. The worldwide sales of the top selling antidepressants are approximately $6.8 billion. As people experience adverse side effects from prescription antidepressants, there has been a concomitant rise in the use of SJW and other herbs as natural antidepressants. Several herbal formulations, purporting to be natural substitutes for Prozac, are already being marketed.

In vitro and in vivo studies have demonstrated that Hypericum constituents may possess antiviral properties. In vitro studies suggest that Hypericum constituents have antiviral activity against cytomegalovirus, herpes simplex, human immunodeficiency virus type 1, influenza virus A, Moloney murine leukemia virus, and sindbis virus. One in vivo study in mice found that low doses of hypericin and psuedohypericin prevented retroviral-induced diseases. Another in vivo study in humans to treat HIV patients with intravenous hypericin was stopped early because light-skinned volunteers developed severe phototoxicity (Botanical Monograph Series, U.S. Pharmacopoeia, March, 1998). More recently, scientists have reported in vivo drug interactions between St. John's Wort extracts and protease inhibitors in HIV patients, and between SJW extracts and cyclosporin in heart transplant patients (Lanclet, 1999).

The National Institutes of Health has initiated a long-term, multicenter, double-blinded clinical trial to study the effects of a Hypericum extract (IL-160 manufactured by Kira), a placebo, and a selective serotonin re-uptake inhibitor (Zoloft) for the treatment of major depression. IL-160, the most studied extract of Hypericum, is standardized to 0.3 wt. percent content of hypericin. Hypericin, as discussed below, may not however be the bioactive ingredient of Hypericum responsible for its anti-depressive activity.

We are utilizing supercritical fluids and near-critical fluids with or without polar cosolvents such as alcohols (SuperFluids™) to improve the quality and manufacturing of St. John's Wort. SuperFluids™ are gases such as carbon dioxide which when compressed, exhibit enhanced thermodynamic properties that can be “fine-tuned” for rapid and selective extraction of bioactive molecules. Such fluids provide the opportunity for more rigorous standardization of nutraceuticals and potential herbal Rₓ products such as St. John's Wort and for achieving formulations that may not be possible with conventional organic phase manufacturing. These products will also be free of toxic organic solvents, environmentally-friendly and truly “green.”

To date, we have identified and characterized an improved SuperFluids™ SJW fraction that can be manufactured in a single-step, solvent free process. This SJW fraction has been shown in an in vitro serotonin reuptake inhibition assay to exhibit a linear dose-response curve as shown in FIG. 1. The bioassay was performed in quadruplicate by Paracelsian, Inc., Ithaca, N.Y. and were all in compliance with our standard operating protocols, having a standard deviation less than ±5%.

We determined by HPLC analysis that the major component of the bioactive SJW 19-A1 fraction was hyperforin with absolute and chromatographic purities of 65.0% and 64.2%. This has been confirmed utilizing internal and external hyperforin standards, and mass spectra analysis by an independent analytical firm (M-Scan, Inc., West Chester, Pa.). Both the HPLC and mass spectra indicated that the bioactive SJW fractions were free of hypericin.

It can be calculated from the data in FIG. 1 that approximately 20.8 mg of the SJW 19-A1 fraction (equivalent to 13.54 mg of hyperforin) is required to establish a 50% serotonin reuptake inhibition level, a typical target for 10 mg doses of Prozac and Zoloft. The SJW fraction, thus, appear to be comparable with commercial antidepressants on an absolute hyperforin mass basis. Comparatively, the percentage serotonin reuptake inhibition for 300 mg of a commercial SJW product (Perika) was 31.7%.

To further confirm our findings that hyperforin may be the bioactive component producing the antidepressant activity in SJW, serotonin reuptake assays were conducted on relatively pure hyperforin with an HPLC chromatographic purity of ˜95% and an absolute purity in excess of 90% [manufactured by Aphios Corporation]; hypericin with an HPLC chromatographic purity ˜85% [Product No. H9252 obtained from Sigma Chemicals]; a commercial hyperforin-based SJW reference product [Perika tablet]; and imipramine hydrochloride with an absolute purity of 99.9% [Product No. 10899, also obtained from Sigma Chemicals]. The latter is an antidepressant with a preference for the norepinephrine transporter but which also blocks serotonin reuptake. The data is summarized in Table 1 below:

TABLE 1
Serotonin Reuptake Inhibition of Relatively
Pure Hyperforin and Hypericin

| | | Serotonin Reuptake |
|---|---|---|
| Sample Description | Sample Weight | Inhibition (%) |
| Blank Control | 0.0 | 0.0 |
| Reference SJW Product (Perika) | 300 mg | 28.2 |
| Imipramine (Sigma) | 65 ng | 38.3 |
| Hyperforin (Aphios) | 2 μg | 80.3 |
| Hypericin (Sigma) | 2 μg | 0.0 |

The data in Table 1 strongly suggests that hyperforin is the bioactive ingredient in serotonin re-uptake inhibition and that hypericin does not have any impact on this antidepression mechanism.

In experiments, we have observed that hyperforin in the SJW fraction is much more stable than the purified hyperforin. Presumably, the increased stability of hyperforin may be from the co-extraction of flavonoids and other components in the SuperFluids™ extraction process, or the serotonin reuptake activity may be the result of another chemical entity, which quantitatively co-elutes with hyperforin.

In other independent studies (TSRL, Inc.), marker compound (hyperforin) and other pharmacophore solutions were prepared below their solubility limit (approximately 5 to 10 μg/ml solution) in perfusion buffer containing 10 mM HEPES, 135 mM NaCl and 5 mM KCl and perfused through rat jejunal intestinal segments at flow rate of 0.2 mL/min. Marker compound concentrations in the perfusion buffer were determined by HPLC analysis. The ratio of the concentration of marker compound in the outflow (after passage through the intestinal segment) vs. the starting concentration is related to the permeability of the compound. In the analysis, we also included metoprolol and carbamazapine (Cbmz) as high permeability standards.

Permeability measurements were made, using the in situ single pass perfusion technique, to determine absorption of hyperforin and other dietary supplements from solutions perfused through jejunal segments in the rat model (Subramanian et al., 1989). Briefly, for each preparation tested, four Sprague-Dawley rats were fasted for 18 hours prior to the study with free access to water. After anesthetizing the animal, its abdomen was opened by a midline incision, a 10 cm jejunal intestinal segment was cannulated at both ends and perfused with the herbal extract solution. The outlet over inlet concentration ratio defines the fraction of drug lost from the solution at steady state and allows estimation of the intestinal permeability (Amidon et al., 1995 and Amidon et al., 1998). Sample analyses were performed by HPLC analysis. The effective intestinal permeability (P_{eff}) was estimated from the steady-state samples utilizing a laminar flow model.

$${P}_{eff} = \frac{Q ( 1 - \frac{{C}_{out}^{′}}{{C}_{i n}^{′}} )}{2 \partial rL}$$

with P_{eff}=effective permeability (cm/sec); Q=flow-rate (ml/min); r=intestinal radius (cm); L=length of the intestinal segment perfused (cm); Cᵢₙ′=inlet concentration normalized for water-flux; and Cₒᵤₜ′=outlet concentration normalized for water-flux. Permeability estimates are calculated from steady-state data points (30-90 minutes) with less then 1%/cm water transport. Our findings on the intestinal permeability of hyperforin and other supplements are plotted in FIG. 2.

Based on the data in FIG. 2, the SuperFluids™ hyperforin extract is very permeable, perhaps 5 to 6 times higher permeability than metoprolol, which is recommended as the “high permeability” internal standard by the FDA in the BCS guidance. It thus appears that the primary challenges for hyperforin are solubilization and stabilization. Permeability is a good indicator for the amount of the bioactive material absorbed; increased solubilization will lead to increased bioactive material absorbed. The SuperFluids™ SJW (hyperforin) extract will be most likely classified as BCS 2, high permeability and low solubility, by the FDA.

The SJW fraction for the preformulation and formulation studies was prepared from fresh Hypericum perforatum biomass in Aphios' SuperFluids™ CXP pilot plant. The extract was chemically analyzed for hyperforin, adhyperforin and hypericin content, and biologically assayed for serotonin re-uptake inhibition. The extract was stored at −20° C. until needed for further use in preformulation and formulation studies.

HPLC assays were conducted on St. John Wort's fractions using a Waters Model 996 HPLC system with photodiode array detector. For hyperforin and adhyperforin, assays will be conducted on a MetaChem C18 column (25 cm×4.6 mm, 5 micron packing) or equivalent with a 90% acetonitrile/H₂O mobile phase to which is added 500 microliters of a 5% (v/v) aqueous solution of 85% phosphoric acid per liter. The flowrate was set to 1.5 ml/min and absorbance will be monitored continuously from 200 nm to 395 nm using a Waters Photo-Diode Array Detector in contour plot mode. Simultaneously, standard chromatographic scans will be obtained using a wavelength of 265 nm.

For hypericin and psuedohypericin, HPLC assays were conducted on a MetaChem C18 column (25 cm×4.6 mm, 5 micron packing) or equivalent with an acetonitrile/methanol/0.1N ammonium acetate, 50:30:20 (v/v/v), mobile phase. The flowrate was set to 1.5 ml/min and absorbance will be measured using a wavelength of 590 nm.

Viscosity was measured using standard rheological methodology, USP 24, NF 19, 2000, <911>. Water Content was measured using Karl Fischer Analysis. Approximately 50 mg of extract was weighed in triplicate transferred to individual 10×75 mm test tubes. These samples were quantitatively transferred into the Karl Fischer sampling port and the moisture content was determined using an EM Science AquaStar C3000 Titrator following the manufacturers recommended procedure. The anode solution was Coulomat A from EM Science. The cathode solution was Coulomat C, EM Science.

Stability studies were carried out in an environmental test chamber (Lab-Line Model 702).

Standard stability studies were conducted on samples, which had passed the accelerated stability studies. The standard studies included 8 time points: t=3, 6, 9 and 12 months at −20° C., 4° C. and 25° C. and t=1, 2, 3, and 6 months at 40° C./75% RH.

In vitro aqueous solubility of test extracts was determined in dissolution media at different pH and surfactant concentrations. The pH solubility over the physiological pH range (1 to 8) was examined. Also a wide range of surfactants (SDS, bile salt mixtures, chremophores) and surfactant concentrations were tested to assess their effects on solubility. For these solubility measurements, an excess of extract or compound was incubated in 10 ml of the aqueous solution and maintained at 37° C. in a shaking water bath. Solubilization of the selected markers was determined over a 24 hour time period. Samples were collected, filtered through syringe filters (0.45 μm pore size), properly diluted and assayed by HPLC. For comparative optimization purposes, solubility in gastric and intestinal fluid collected from dogs can also be determined. Gastric fluid is generally clear or slightly yellow and viscous, with a pH between 1 and 3. Intestinal fluid is also viscous, but having a bright yellow appearance due to the presence of bile salts. The pH of this fluid is in the range of 5 to 6.5. For solubility measurement in in vivo fluids, approximately 2 ml of fluids (gastric or intestinal) were placed in microcentrifuge tubes and maintained at 37° C. in a shaking water bath. Extract amounts exceeding the expected solubility of a given marker compound were placed in the tubes. Solubilization of the selected markers was determined over a 24 hour time period. Samples were collected, filtered through syringe filters (0.45 μm pore size), properly diluted and assayed by HPLC.

Solubility—Organic Solvents and Oils:

For these solubility measurements, an excess of extract or compound was incubated in 10 ml of the neat solvent or oil and maintained at 37° C. in a shaking water bath. Potential solvents and oils are listed in Table 2.

TABLE 2
Potential Solvents and Oils to be Tested

| | |
|---|---|
| Castor oil | Stearic acid |
| Gelucire | Stearyl alcohol |
| Hydrogenated vegetable oil | Ethanol |
| Lecithin | Methanol |
| Maize oil | Isopropyl alcohol |
| Olive oil | Soya oil |
| Paraffin oil | |

Solubilization of the selected markers was determined over a 24 hour time period. Samples were collected, filtered through syringe filters (0.45 μm pore size), properly diluted and assayed by HPLC.

Excipient Compatibility and Stability Study: The SJW fraction was systematically screened with various combinations of GRAS excipients suitable for oral administration. The goal was to assess the longevity of these solutions and their stabilities relative to freshly made samples of the existing formulation. We evaluated antioxidants such as Vitamin E at different concentrations in the 0.1 to 1.0% range, together with bulking agents such as PEG 400, soybean oil and other oils. In formulation development consideration was also given to the costs of excipients and manufacturing, and their impact on final product cost. We evaluated the use of variations of novel formulations containing natural mixed tocopherols, vitamin E, vitamin E-TPGS, d-α-tocopheryl, polyethylene glycol 1000 succinate for improving stability. Table 3, column 1 lists the excipients and solvents evaluated. Column 2 of the table identifies the ratio of SJW fraction to excipient to be evaluated. The fraction alone served as a control.

TABLE 3
Typical Excipients (not inclusive) to
be used for Compatibility Studies

| | Ratio |
|---|---|
| Component | (SWJ Fraction:Excipient) |
| Vitamin E-TPGS | varied |
| Hydroxypropylmethyl Cellulose | 1:1 |
| Lactose Monohydrate (NF) | 1:1 |
| Sorbitol (NF) | 1:1 |
| Microcrystalline Cellulose (NF) | 1:1 |
| Sodium Chloride (USP) | 1:1 |
| Sodium Starch Glycholate (NF) | 10:1 |
| Colloidal Silicon Dioxide (NF) | 10:1 |
| Magnesium Stearate (NF) | 10:1 |
| Starch (NF) | 1:1 |
| Povidone (NF) | 1:1 |
| Ethylcellulose (NF) | 1:1 |
| Hydroxypropyl Cellulose (NF) | 1:1 |
| Talc (USP) | 1:1 |
| Crospovidone (NF) | 1:1 |
| Ethanol (USP) | |
| Isopropyl Alcohol (USP) | |

The samples were prepared by mixing pure SJW extract using a geometric dilution technique in glass scintillation vials under ambient conditions. Extract material may also be prepared as a wet granulation in a mortar and pestle. Finished granulations will be packaged in 20 ml glass scintillation. Samples will be stored in sealed 20 ml scintillation vials. A separate vial will be prepared for each test interval. Two storage temperatures will be used 25° C. (control temperature) and 40° C. (elevated temperature). At each test interval samples will be visually examined for physical change and hyperforin content by HPLC. The proposed test intervals are 0, 2, 4, 6, 8, and 12 weeks. Original data will be presented in a table and/or a graph format. Statistical analysis of the data sets will be performed using the appropriate statistical model (s) and software (e.g., SYSTAT®).

Formulation Studies to Develop Prototype Dosage Forms

As indicated in the preliminary data, the permeability of the hyperforin is very high, so the challenge for this formulation is to maintain stability and achieve rapid dissolution. As such, the goal was to establish two formulations: (1) a hard tablet and (2) a liquid formulation that would be suitable for capsules. The formulation development process is an iterative process, where a range of formulations is tested for appropriate characteristics, including stability of the SJW fraction (hyperforin) and rapid dissolution of the bioactive hyperforin from the dosage form. We anticipate that the prototype formulations that are developed here would be suitable for in vivo studies in animal models and, subsequently, in clinical trials.

Tablet Type Formulation: Following the excipient compatibility studies, a variety of lab scale wet granulations using compatible excipients were tested using standard pharmacy practices (e.g., raw materials are mixed by geometric dilution, mixing is performed using a mortar and pestle and spatula). Typical batch sizes were on the order of 3 grams. A target SWJ fraction weight was determined based on content of hyperforin. Wet granulations were tested for particle size, size distribution, compressibility, bulk and tap density, hardness, disintegration and dissolution following standard operating procedures. Potential tablet formulations were tested for enteric coating.

Particle size and size distribution will be determined using the Aerosizer LD (Amherst Process Instruments, Inc.) following the manufacturer's recommended procedures. We will utilize standard operating procedures for the determination of density, hardness, disintegration properties following USP/NF guidelines and standard pharmaceutical practices. Different polymer coatings (e.g., enteric polymers) will be tested on the bench scale in the event that there is acid instability. Typical polymers include cellulose acetate phthalate and related compounds and the Eudragit series of polymers. These will be tested for dissolution under gastric and intestinal conditions.

Liquid Type Formulation: Potential liquid formulations will be evaluated based on the solubility/stability of the SJW extract and hyperforin in oil. Formulations will be stability tested in the presence and absence of various concentrations of vitamin E-TPGS.

In Vitro Dissolution: Ideally, an in vitro dissolution testing should reflect the in vivo solubilization conditions. Real in vivo conditions are complex and may include particle-particle interaction that leads to particle aggregation, position dependent permeability and metabolism, changing pH, luminal content, hydrodynamics in the GI tract. Thus, in vitro dissolution test may include multiple dissolution medium, multiple time points, surfactants, and varying mixing speeds to reflect these complex in vivo conditions. Using the solubilization results, we developed dissolution methodology

Dissolution testing was done with USP Apparatus 2 or USP Apparatus 3 machines. Typical conditions for dissolution rate determination for Apparatus 2 are given below as an example.
USP Apparatus 2 (set to paddle depth)
Paddle rate—100 RPM
Temperature—37° C. (+0.50° C.)
Vessel size—900 ml
Vessel volume—500 ml
Dissolution media—SIF, +/− surfactants, etc.
Sample points—0, 10, 20, 30, 60, 90, 120 min
Sample volume—3 ml
Replacement volume—3 ml
Filter—Gelman GHP Acrodisc 0.45 μm 13 mm

The paddles are lowered into the dissolution media and the test is initiated. At the designated intervals samples are withdrawn from the dissolution and filtered. The filtered samples are assayed neat by HPLC or LC/MS/MS. Both pH and surfactant concentration of the media will be changed to study the effects of the parameters on the release rate. Different surfactants will be used including SLS, Cremophor EL, and Tween 80. The amount of active compounds in the formulations will be determined by considering solubility of drugs so that the sink conditions are maintained in the dissolution media. Dissolution testing will also be determined in apparatus 3, which has the advantage of ease of media changes to better reflect in vivo conditions.

The results of the experiments were be used to design a SJW formulation for the manufacturing of a SJW product. Based on the very hydrophobic nature and waxy nature of the SJW fraction, the fraction may best be delivered in a softgel or vegetable capsule. The major issue with the use of these technologies is often problems with oxidation-sensitive compounds, as the softgel capsule shell is permeable to oxygen. This problem was successfully countered with use of GRAS antioxidants such as natural vitamin E and natural mixed tocopherols. Additionally, a less permeable vegetable capsule was utilized and the formulation was capped with a nitrogen head to displace any air (oxygen) from the capsule as a further measure to protect the product from oxidative damage. Product oxidation liability was assessed by RP-HPLC and/or LC/MS when the product is exposed and stored in these capsules. We also evaluated the compatibility between the SJW fraction, excipients and gel capsules. Compatibility was evaluated in terms of loss of integrity or stability of any of the components, measured by HPLC.

Preferably, the formulation can be maintained at a slightly acidic pH below 4.8, which is the pKa of hyperforin.

Preferably, more stable salts of hyperforin can be utilized in the formulation.

A method for formulating fractions of Hypericum comprising different active ingredients or proportions of active ingredients is desired. Products incorporating such fractions could be marketed for different indications.

### SUMMARY OF THE INVENTION

Embodiments of the present invention are directed to the methods of formulating fractions of Hypericum. The formulations can be used to make St. John's Wort products.

Aspects of the present invention employ materials known as supercritical, critical or near-critical fluids. A material becomes a critical fluid at conditions that equal its critical temperature and critical pressure. A material becomes a supercritical fluid at conditions that equal or exceed both its critical temperature and critical pressure. The parameters of critical temperature and critical pressure are intrinsic thermodynamic properties of all sufficiently stable pure compounds and mixtures. Carbon dioxide, for example, becomes a supercritical fluid at conditions that equal or exceed its critical temperature of 31.1° C. and its critical pressure of 72.8 atm (1,070 psig). In the supercritical fluid region, normally gaseous substances such as carbon dioxide become dense phase fluids that have been observed to exhibit greatly enhanced solvating power. At a pressure of 3,000 psig (204 atm) and a temperature of 40° C., carbon dioxide has a density of approximately 0.8 g/cc and behaves much like a nonpolar organic solvent, having a dipole moment of zero debyes. A supercritical fluid uniquely displays a wide spectrum of solvation power as its density is strongly dependent upon temperature and pressure. Temperature changes of tens of degrees or pressure changes by tens of atmospheres can change a compound's solubility in a supercritical fluid by an order of magnitude or more. This unique feature allows for the fine-tuning of solvation power and the fractionation of mixed solutes. The selectivity of nonpolar supercritical fluid solvents can also be enhanced by addition of compounds known as modifiers (also referred to as entrainers or cosolvents). These modifiers are typically somewhat polar organic solvents such as acetone, ethanol, methanol, methylene chloride or ethyl acetate. Varying the proportion of modifier allows a wide latitude in the variation of solvent power.

In addition to their unique solubilization characteristics, supercritical fluids possess other physicochemical properties that add to their attractiveness as solvents. They can exhibit liquid-like density yet still retain gas-like properties of high diffusivity and low viscosity. The latter increases mass transfer rates, significantly reducing processing times. Additionally, the ultra-low surface tension of supercritical fluids allows facile penetration into microporous materials, increasing extraction efficiency and overall yields.

While similar in many ways to conventional nonpolar solvents such as hexane, it is well-known that supercritical fluid solvents can extract a different spectrum of materials than conventional techniques. Product volatilization and oxidation as well as processing time and organic solvent usage can be significantly reduced with the use of supercritical fluid solvents.

A material at conditions that border its supercritical state will have properties that are similar to those of the substance in the supercritical state. These so-called “near critical” fluids are also useful for the practice of this invention. For the purposes of this invention, a near critical fluid is defined as a fluid which is (a) at a temperature between its critical temperature (T_{c}) and 75% of its critical temperature and at a pressure at least 75% of its critical pressure, or (b) at a pressure between its critical pressure (P_{c}) and 75% of its critical pressure and at a temperature at least 75% of its critical temperature. In this definition, pressure and temperature are defined on absolute scales, e.g., Kelvins and psia. Table 4 shows how these requirements relate to some of the fluids relevant to this invention. To simplify the terminology, materials that are utilized under conditions that are supercritical, near critical, or exactly at their critical point will jointly be referred to as “SCCNC” fluids.

TABLE 4
Physical Properties of Critical Fluid Solvents

| | | BP | Pᵥₐₚ | T_{c} | P_{c} | 0.75T_{c} | 0.75P_{c} |
|---|---|---|---|---|---|---|---|
| Fluid | Formula | (° C.) | (psia @ 25° C.) | (° C.) | (psia) | (° C.) | (psia) |
| Carbon dioxide | CO₂ | −78.5 | 860 | 31.1 | 1070 | −45.0 | 803 |
| Nitrous oxide | N₂O | −88.5 | 700 | 36.5 | 1051 | −41.0 | 788 |
| Propane | C₃H₈ | −42.1 | 130 | 96.7 | 616 | 4.2 | 462 |
| Ethane | C₂H₆ | −88.7 | 570 | 32.3 | 709 | −44.1 | 531 |
| Ethylene | C₂H₄ | −103.8 | NA | 9.3 | 731 | −61.4 | 548 |
| Freon 11 | CCl₃F | 23.8 | 15 | 198.1 | 639 | 80.3 | 480 |
| Freon 21 | CHCl₂F | 8.9 | 24 | 178.5 | 750 | 65.6 | 562 |
| Freon 22 | CHClF₂ | −40.8 | 140 | 96.1 | 722 | 3.8 | 541 |
| Freon 23 | CHF₃ | −82.2 | 630 | 26.1 | 700 | −48.7 | 525 |

Table 4 Notes:
BP = Normal boiling point;
Pᵥₐₚ = Vapor pressure

Embodiments of the present invention are directed to methods of making fractions of Hypericum. One method comprising the steps of contacting a Hypericum biomass with a first solvent comprising a critical, super critical or near critical fluid, to allow one or more first constituents of said Hypericum biomass to dissolve into the first solvent. The method further comprises the step of separating the first solvent from the Hypericum biomass to form a first fraction. The method further comprises the step of contacting the Hypericum biomass with at least one subsequent solvent comprising a critical, super critical or near critical fluid, to allow one or more additional constituents of the Hypericum biomass to dissolve into the subsequent solvent. The subsequent solvent is separated from said Hypericum biomass to form at least one subsequent fraction. The first solvent and at least one of the subsequent solvents have different solvation properties. The solvation properties are different due to at least one difference in one of the parameters of material of the first and subsequent critical, supercritical or near critical fluid, temperature, pressure, or concentration of entrainers and modifiers. Finally, the critical, supercritical and near critical fluid is removed from at least one of said first or subsequent fractions to form at least one concentrated fraction extract.

Preferably, each subsequent solvent is altered to change the solvation properties of the extracting fluid, so that each step can recover a different spectrum of compounds. The solvation properties of SCCNC fluids can be altered by changing the temperature or pressure of the fluid. By way of example, a preferred temperature and pressure for a SCCNC comprising carbon dioxide is a temperature in the range of 10 to 60° C. and a pressure in the range of 1,000 to 5,000 psig.

Preferred SCCNC fluids comprise carbon dioxide, nitrous oxide, ethylene, ethane, propane and fluorohydrocarbons. The fluid may also contain modifiers. Preferred modifiers are methanol, ethanol, propanol, butanol, methylene chloride, ethyl acetate and acetone.

A preferred modifier comprises methanol. In one preferred embodiment, each subsequent extraction employs a larger concentration of methanol. Thus, the plurality of solvents becomes increasingly more hydrophilic. The first extraction step tends to remove lipophilic compounds while the last extraction step tends to remove hydrophilic compounds. Removal of the lipophilic materials allows the next more hydrophilic critical fluid to have access to more hydrophilic compounds trapped in cellular structures. Preferred methanol concentration ranges, based on carbon dioxide at a pressure of 3000 psig and a temperature of 40° C., are 0-5 volume %. For the same temperature and pressure, 5-10 volume % methanol is preferred for a second extraction step; 10-20 volume % methanol is preferred for a third extraction step; 20-30 volume % methanol is preferred for a fourth extraction step; 30-50 volume % methanol is preferred for a fifth extraction step.

Surprisingly and unexpectedly, the sequential extraction with varying polarity solvents produces larger numbers of fractions exhibiting better biological activity and stability than corresponding fractions derived from conventional organic solvent extractions. The use of SCCNC fluids allows for easy removal of much of the solvent by mere depressurization. Use of a single apparatus to perform the sequential extraction or fractionation steps minimizes labor and increases efficiency. Indeed, the entire process can be readily automated. The use of SCCNC fluids allows the extraction conditions to be readily varied by temperature, pressure, or modifier solvents, minimizing equipment needs, processing time, potential for contamination, and loss of yield. These and other features and advantages will be readily apparent from the drawing and detailed discussion which follow.

The hyperforin-enriched Hypericum fraction can then be solubilized at specific concentration in a pharmaceutically acceptable excipient such as Extra Virgin olive oil. The concentration of the hyperforins (hyperforin and adhyperforin) in the excipient can be specified to establish a pre-specified level of serotonin reuptake inhibition per the linear regression curve in FIG. 1. As such the formulation can be standardized on the basis of chemical composition to achieve a pre-specified level of biological activity.

Antioxidants can be added to the formulation to act as an oxygen scavenger and improve the stability of the hyperforins. Antioxidants can include Vitamin E and natural mixed tocopherols. Excess antioxidants in the formulation would provide an additional nutritional benefit to the recipient.

Emulsifiers such as lecithin can be added to the formulation to increase the solubility and bioavailability of the water insoluble hyperforins that are already highly permeable as shown in FIG. 2.

Additionally agents can be added to increase the viscosity and adsorb moisture to protect the capsule and improve manufacturability. Such agents include silicon dioxide and titanium oxide.

The formulation can be encapsulated in gelatin capsules or, preferably, vegetable capsules. The capsules can be topped with a head of nitrogen to remove any excess oxygen that may have a deleterious effect on the product.

The capsules can be packaged in amber bottles or UV resistant plastic bottles to protect them from light and stored in a cool, dry place or preferable in a refrigerator between 4 and 10° C. to maximize stability.

### BRIEF DESCRIPTION OF THE DRAWINGS

FIG. 1 is a regression plot of serotonin reuptake inhibition of SCCNC (SuperFluids) SJW fraction.

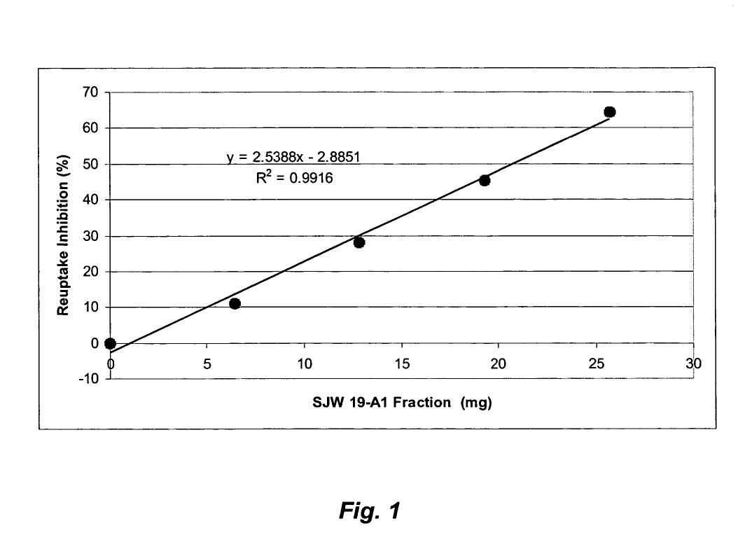

FIG. 2 is a bar graph showing intestinal permeability of hyperforin and other dietary supplement marker compounds.

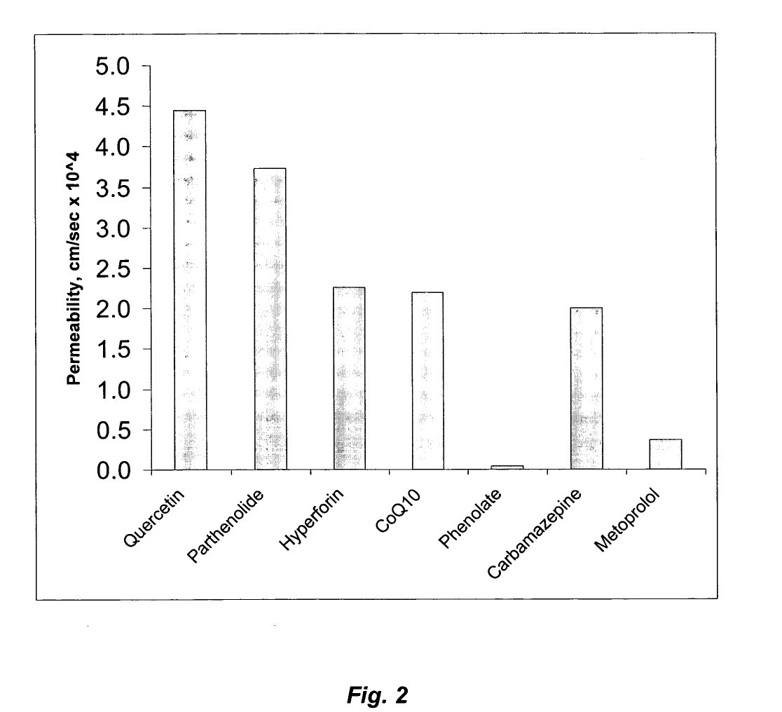

FIG. 3 is a flow scheme for the SCCNC fractionation/extraction apparatus used in the examples of this specification.

FIG. 4 is a bar graph showing the biological activities of SCCNC fractions obtained through the practice of this invention.

### DETAILED DESCRIPTION OF PREFERRED EMBODIMENTS

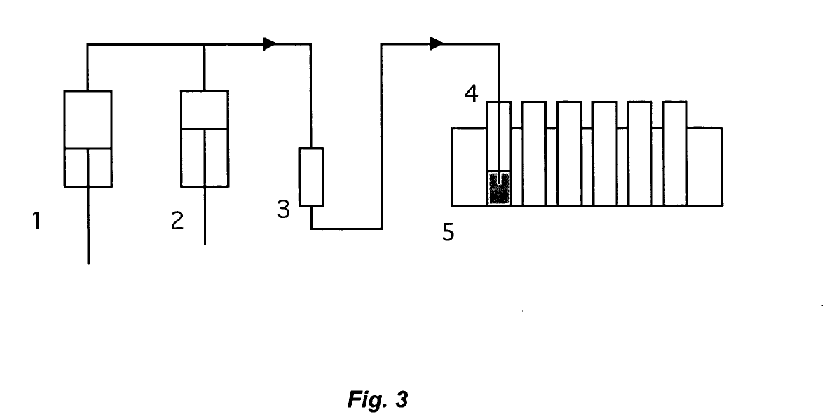

SCCNC fluid fractionation can be carried out on an ISCO (Lincoln, Nebr.) SFX 3560 automated extractor or a manual version of the same. As shown in FIG. 3, this is a dual pump system, utilizing syringe pump 1 for neat critical fluid and syringe pump 2 for modifier. The pumps are independently controllable, allowing easy adjustment of the fluid composition. To prepare a sample, the Hypericum biomass was dried between 40 and 60° C. for 1 day, with or without vacuum and ground into a fine powder (around 100 mesh). The dried powder was transferred to a 10 ml ISCO extraction cartridge, numbered 3 in the figure, after which the cartridge was optionally filled with glass wool or cotton to reduce the dead volume. After loading a cartridge on the cartridge holder, the sequential extraction/fractionation procedure was commenced. The system was brought to 3,000 psig and 40° C., and extracted for 30 minutes with pure CO₂. This fraction was collected in methanol in a glass vial, numbered 4 in the figure.

Next, the extraction parameters were set to: Supercritical CO₂ at 3000 psig and extraction temperature 40° C., step extractions with methanol as cosolvent at 5, 10, 20, and 40 vol %, each step being 10 min. Because some void volume remained, the composition of the extraction medium did not change sharply or immediately when modifier flowrate was adjusted to give a new fluid composition. Each sample thus yielded 5 fractions, which were collected in methanol in separate glass vials. The different collection vials are mounted in a carousel, numbered 5 in the figure. The vials are automatically positioned by the SFX 3560 extractor apparatus. While the preceding steps were carried out in a continuous flow mode, cessation of flow to allow static contact time is also contemplated. This procedure may allow a reduction in the amount of extraction solvent required.

Several different experiments were conducted to evaluate the SCCNC fluids fractionation of Hypericum biomass and formulating the hyperforin-enriched Hypericum fractions into resulting St. John's Wort products.

### EXAMPLES

### Example 1

### Fractionation of Hypericum Biomass with SCCNC Fluids

Dried Hypericum biomass (Lot # 335H699116), obtained from Wilcox Natural Products, Boone, N.C., was separated from twigs and branches. This material was ground to a fine powder. Three grams of dried and ground Hypericum biomass was fractionated with supercritical carbon dioxide and methanol at 3,000 psig and 40° C. The fractionation was carried out initially with neat carbon dioxide and then by incrementally adding methanol to increase the polarity of the working solvent. The extraction was carried out in an apparatus similar to that shown as FIG. 3. The fractions were dried under vacuum at approximately 40° C. for 18 hours. The results of the fractionation are shown in Table 5 below:

TABLE 5
Fractionation of Hypericum Biomass with
SCCNC Fluids Carbon Dioxide/Methanol

| | | Amount | Percentage |
|---|---|---|---|
| | | Extracted | Extracted |
| Fraction | Description | (mg) | (%) |
| SJW-2A | Carbon Dioxide with 0% Methanol | 95.0 | 3.17 |
| SJW-2B | Carbon Dioxide with 5% Methanol | 16.6 | 0.55 |
| SJW-2C | Carbon Dioxide with 10% Methanol | 27.1 | 0.90 |
| SJW-2D | Carbon Dioxide with 20% Methanol | 90.8 | 3.01 |
| SJW-2E | Carbon Dioxide with 30% Methanol | 4.5 | 0.02 |
| SJW-2F | Carbon Dioxide with 40% Methanol | 6.0 | 0.02 |

### Example 2

### Biological Activity of SCCNC Fluids St. John's Wort Fractions

The first four fractions in Experiment SJW-2 in Example 1 above were dissolved in DMSO to 5 mg/ml; no insoluble matter was observed.

Rat brain synaptosomes were prepared from the cortex for ³H-5-HT uptake. Male Sprague-Dawley rats were decapitated and the brains were rapidly removed. Cortices were weighed and homogenized in 9 volumes of ice-cold 0.32M sucrose solution using a Potter-Elvejhem homogenizer. The homogenate was centrifuged at 1,000 g at 4° C. for 10 min. The supernatant was decanted and used for uptake experiments.

Fifty μl aliquots of the crude synaptosomal preparations were incubated in 1.2 ml of incubation medium at 37° C. of the following composition (mM concentrations): NaCl 109, KCl 3.55, CaCl₂ 2.4, MgSO₄ 0.61, KH₂PO₄ 1.1, NaHCO₃ 25, glucose 5.4, nialamide 0.025, pH 7.4 (this medium was gassed with 95% O₂-5% CO₂, 30 min prior to use) with ³H-5-HT. An incubation period of 5 min was employed. The uptake was stopped by dilution with 1.5 ml of ice-cold medium followed immediately by filtration under reduced vacuum through Whatman GFIB glass fiber filters. The filters were washed twice with 3 ml of ice-cold medium and dried. After addition of scintillant cocktail, ³H-radioactivity was counted. Stock solutions of test compounds prepared in DMSO, were centrifuged at 17,000×g for 10 min and the supernatants were used in the assays. Further dilutions were made in incubation medium.

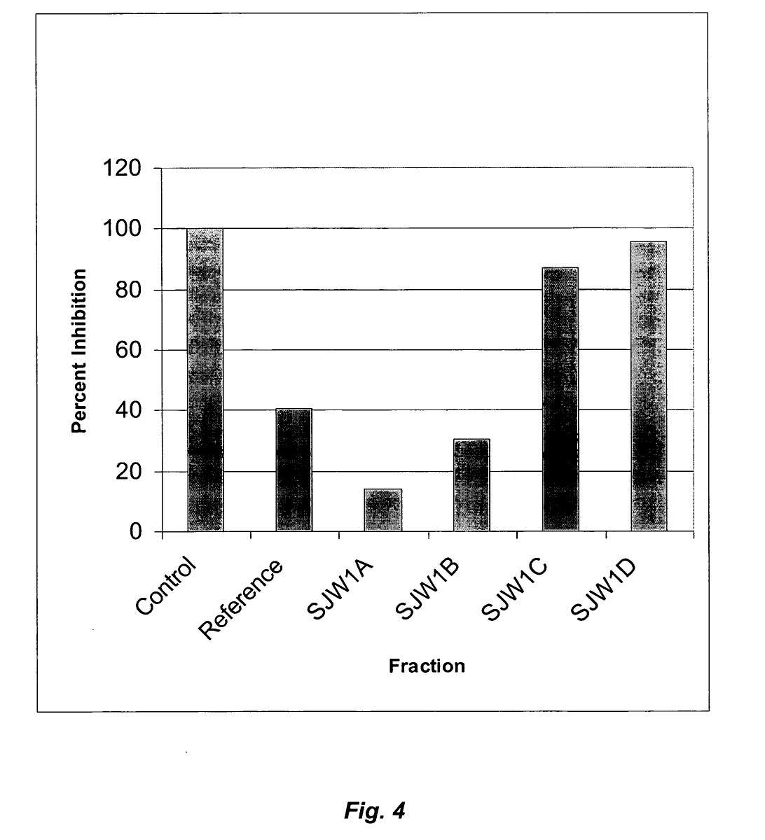

Samples were assayed in four replicates at a single dose of 16.7 μg/ml and compared to a reference extract (Perika tablets, Nature's Way St. John's Wort, Bar Code 33674-06560, Lot 710042, Expiration October 2000) at the same concentration in a serotonin specific re-uptake assay by Paracelsian, Ithaca, N.Y. This assay evaluates the re-uptake or the re-uptake inhibition (1-re-uptake) of radiolabeled serotonin taken up into a neural (syntaptosome) preparation in the presence of St. John's Wort fractions. The results are listed in Table 6 and shown in FIG. 4.

TABLE 6
Inhibition of Serotonin Re-Uptake by SCCNC
Fluids St. John's Wort Fractions

| | | | Serotonin |
|---|---|---|---|
| | | Mean | Re-Uptake |
| Fraction | Description | Counts | (%) |
| Control | DMSO | 24,108 | 100.0 |
| Reference | Extract of Perika tablets | 9,792 | 40.6 |
| SJW-2A | Carbon Dioxide with 0% Methanol | 3,379 | 14.0 |
| SJW-2B | Carbon Dioxide with 5% Methanol | 7,381 | 30.6 |
| SJW-2C | Carbon Dioxide with 10% Methanol | 20,998 | 87.1 |
| SJW-2D | Carbon Dioxide with 20% Methanol | 23,140 | 96.0 |

SCCNC fluids fractions SJW-2A and SJW-2B are extremely potent compared to the reference Perika product. The first fraction SJW-2A is about three times more potent than the reference Perika tablets at the same concentrations.

### Example 3

### Chemistry of SCCNC Fluids St. John's Wort Fractions

HPLC assays were conducted on several SCCNC fluids St. John Wort's fractions, and a methanol extract of several Perika tablets. The assays were conducted with a MetaChem C18 column (25 cm×4.6 mm, 5 micron packing) and a 90% acetonitrile/H₂O mobile phase with 500 microliters of a 5% (v/v) aqueous solution of 85% phosphoric acid per liter. The flowrate was 1.5 ml/min. Absorbance was monitored continuously from 200 nm to 395 nm using a Waters Photo-Diode Array Detector in contour plot mode. Simultaneously, standard chromatographic scans were obtained using a wavelength of 265 nm.

The SCCNC fluids St. John Wort's fractions were prepared in the same manner as Examples 1 and 2, by fractionating 3 grams of St. John's Wort with supercritical carbon dioxide and methanol at 3,000 psig and 40° C. The fractionation was carried out initially with neat carbon dioxide and then by incrementally adding methanol to increase the polarity of the working solvent. Sample SJW-3A was extracted from Hypericum biomass with neat carbon dioxide at 3,000 psi and 40° C.; sample SJW-3B was extracted from the same biomass with 95:5:carbon dioxide:methanol at 3,000 psi and 40° C.; sample SJW-3B was extracted from the same biomass with 90:10:carbon dioxide:methanol at 3,000 psi and 40° C.; and sample SJW-3B was extracted from the same biomass with 80:20:carbon dioxide:methanol at 3,000 psi and 40° C.

A methanol extract of St. John's Wort was prepared by extracting 5 grams of St. John's Wort with 100 ml HPLC grade methanol at 50° C. The extraction was conducted with continuous stirring on a magnetic hot plate for more than 2 hours. The extractant was then filtered through a 0.45 micron Whatman filter to give a clear filtrate for analysis. The residue was re-extracted twice and analyzed. No actives were extracted in the subsequent extractions indicating that the first extraction was complete.

Ten Perika tablets (Nature's Way St. John's Wort, Bar Code 33674-06560, Lot 710042, Expiration October 2000) were weighed and ground into a fine powder. The ground tablets were extracted by stirring with 30 ml of 95% water/5% methanol mixture for 40 minutes. The extractant was then brought to 70 ml with a mixture of 92% methanol/8% water and mixed with a magnetic stir bar for 10 minutes. The extractant was filtered through a 0.45 micron Whatman filter and the filtrate transferred to a 100 ml volumetric flask. The solids were rinsed with a mixture of 92% water/8% methanol, and the rinse added to the 100 ml volumetric flask. The extracts were brought up to 100 ml and analyzed. The solids were re-extracted with an additional 12 ml of 92% methanol/8% water and filtered. The filtrate from the second extraction was analyzed but contained no actives. The solids from the second extraction were soaked in 2 ml of water, then mixed with 18 ml of 92% methanol/8% water and filtered. The filtrate from the third extraction was analyzed but contained no actives.

HPLC chromatograms of the first SCCNC fluids St. John Wort's fraction and the methanol extract of Perika tablets were compared. Hyperforin was readily identified from the UV spectrum in the paper by Holzl and Ostrowski (1987) as eluting at 10.3 mins under the HPLC conditions used. The results of the fractionation of St. John's Wort with SCCNC carbon dioxide/methanol are listed in Table 7.

TABLE 7
Chemistry of SCCNC Fluids St. John's Wort Fractions

| | Amount | Percentage | | Hyperforin | Hyperforin |
|---|---|---|---|---|---|
| | Extracted | Extracted | Hyperforin | Absolute | Chromat. |
| Fraction | (mg) | (%) | (mg) | Purity (%) | Purity (%) |
| SJW-3A | 67.9 | 2.3 | 29.7 | 43.7 | 72.7 |
| SJW-3B | 70.9 | 2.4 | 23.1 | 32.6 | 72.8 |
| SJW-3C | 62.9 | 2.1 | 6.4 | 10.2 | 71.6 |
| SJW-3D | 75.6 | 2.5 | 2.6 | 3.4 | 71.0 |
| Total | 277.3 | 9.3 | 61.8 | ˜20.6 | ˜72.0 |

The chromatographic purities of hyperforin in the SCCNC St. John's Wort fractions A through D were between 71 and 73%, while the chromatographic purity of the Perika extract was about 7%.

Approximately 61.8 mg hyperforin was extracted from 3 grams of raw materials, giving a SCCNC fluids fractionation yield of approximately 20.6 mg/gm. A parallel warm methanol extraction of Hypericum biomass yielded approximately 21.0 mg/gram. The SCCNC fluids fractionation yielded about the same amount (approximately 100%) of hyperforin extracted from the same raw materials. However, the absolute (43.7%) and chromatographic purities (72.7%) of hyperforin in the SCCNC fluids St. John's Wort fraction A and the absolute (32.6%) and chromatographic (72.8%) purities of hyperforin in the SCCNC fluids St. John's Wort fraction B were much higher than the absolute (9.0%) and chromatographic (18.9%) purities of hyperforin in the methanolic Hypericum fractions.

### Example 4

### Chemical Solution Stability of SCCNC Fluids St. John's Wort Fractions

SCCNC fluids St. John Wort's Fraction A was analyzed by the assay described in Example 3. After analysis, the vial was re-capped and stored in its methanol solvent in a dark cabinet at room temperature. The sample was then re-analyzed after 11, 50 and 53 days utilizing an identical HPLC procedure. As listed in Table 8, the HPLC profiles and the quantity of the primary component, hyperforin, remain almost unchanged after 11 days in a methanol solution at room temperature. After 53 days, the hyperforin concentration had fallen to about 77% of its initial value without significant deterioration in quality.

TABLE 8
Chemical Solution Stability of SCCNC Fluids Carbon
Dioxide/Methanol St. John's Wort Fraction

| | | | | % Change in |
|---|---|---|---|---|
| | | | Hyperforin | Hyperforin |
| | | | Chromato- | Chromato- |
| Time | Hyperforin | % Change in | graphic | graphic |
| (days) | (ppm) | Hyperforin | Purity (%) | Purity (%) |
| 0 | 994 | 0.0 | 70.0 | 0.0 |
| 11 | 964 | −3.0 | 69.6 | −0.6 |
| 50 | 793 | −20.2 | 67.5 | −3.6 |
| 53 | 763 | −23.2 | 66.3 | −5.3 |

### Example 5

### Biological Stability of SCCNC Fluids St. John's Wort Fractions

Samples of SCCNC fluids St. John Wort's Fractions analyzed in Example 2 were subsequently re-analyzed for biological activities by a serotonin re-uptake assay identical to the assay described in Example 2 above. In the interval between analyses, the samples were stored at 4° C. except for two periods of overnight shipment on ice and several thaw cycles during chemical and biological analyses. The results of the analyses are listed in Table 9.

TABLE 9
Biological Stability of Serotonin Re-Uptake Inhibition
by SCCNC Fluids St. John's Wort Fractions

| | | Serotonin | Serotonin |
|---|---|---|---|
| | | Re-Uptake | Re-Uptake |
| | | (%) time = | (%) time = |
| Fraction | Description | 6 days | 71 days |
| Control | DMSO | 100.0 | 100.0 |
| SJW-2A | Carbon Dioxide with 0% Methanol | 14.0 | 13.5 |
| SJW-2B | Carbon Dioxide with 5% Methanol | 30.6 | 47.6 |
| SJW-2C | Carbon Dioxide with 10% Methanol | 87.1 | 72.8 |
| SJW-2D | Carbon Dioxide with 20% Methanol | 96.0 | 96.4 |

### Example 6

### Formulation of SCCNC Fluids St. John's Wort Fractions

An example formulation of a hyperforin-enriched SJW fraction is listed in Table 10.

TABLE 10
Superantioxidant Formulation of SCCNC
Fluids St. John's Wort Fractions

| | | |
|---|---|---|
| Component | Amount | Daily Value* |

| Hyperforins | 15 | mg | ** |
|---|---|---|---|
| Natural Vitamin E | 6 | IU | 20% |
| Natural Mixed Tocopherols 90% | 50 | mg | ** |
| Total Calories | 4.27 | | 0.21% |
| Total Fat | 0.43 | g | 0.66% |
| Total Carbohydrates | 0.1 | g | 0.03% |

*Daily Value based on 2,000 calorie diet
** Daily Value not established.

Other ingredients include lecithin, olive oil (Extra Virgin), SCCNC Fluids St. John's Wort Fraction, Hypromellose Capsule, Silicon Dioxide.

It is intended that the matter contained in the preceding description be interpreted in an illustrative rather than a limiting sense.

What is claimed is:

1. A composition comprising hyperforin-enriched Hypericum.

2. The composition of claim 1, wherein said hyperforin-enriched Hypericum is solubilized in a pharmaceutically acceptable excipient.

3. The composition of claim 2, wherein said excipient is olive oil.

4. The composition of claim 1 further comprising one or more antioxidants.

5. The composition of claim 4, wherein said antioxidant is Vitamin E.

6. The composition of claim 4, wherein said antioxidant is natural mixed tocopherols.

7. The composition of claim 1 further comprising one or more emulsifiers.

8. The composition of claim 7, wherein said emulsifier is lecithin.

9. A capsule comprising the composition of claim 1.

10. A capsule comprising the composition of claim 1, wherein said capsule is a gelatin capsule.

11. A capsule comprising the composition of claim 1, wherein said capsule is a vegetable capsule.

12. The capsule of claim 9 further comprising one or more hygroscopic agents.

13. The capsule of claim 12, wherein said hygroscopic agent is silicon dioxide.

14. The capsule of claim 12, wherein said hygroscopic agent is titanium oxide.
